(19) United States
(12) Patent Application Publication
(10) Pub. No.: US 2005/0126565 A1
(43) Pub. Date: Jun. 16, 2005

(54) Emergency breathing tube

(76) Inventor: Shang Lung Huang, Taichung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/998,646

(22) Filed: Nov. 30, 2004

(30) Foreign Application Priority Data
Dec. 12, 2003 (TW) 92221910

(51) Int. Cl. A61M 16/00

(52) U.S. Cl. 128/202.13; 128/200.24; 128/200.26

(57) ABSTRACT

A tractable emergency breathing tube for life-saving in the event of fire includes a first tube and a second tube assembled together to form a series of telescoping tubes. The telescoping tubes are nested one within the other when the emergency breathing tube in a retracted stowed position. The inner tubes are latched in the stowed position by a caging mechanism. Furthermore, the emergency breathing tube can be extended until the final deployed position to reached out of the building on fire for fresh air. A user can inhale fresh air through the pursed mouth slowly by means of the instrument and exhale carbon dioxide through the nose rapidly, and repeat the above inhale/exhale procedures until being rescued.

### BACKGROUND OF THE INVENTION

1. Field of the Invention

The present invention relates to an emergency breathing tube for fire victims, and more particularly to a breathing tube that people in a smoky fire scene can utilize to get fresh air from an outdoor location far away from the fire scene, thereby reducing injury and mortality due to inhaling highly poisonous gases, such as carbon monoxide, or due to lack of oxygen.

2. Description of Prior Art

In practice, most fire victims die from inhaling highly poisonous gases, such as carbon monoxide, or a lack of oxygen. Though conventional fire escape systems, for example, emergency elevators, escape ladders and the like may help people to get away from a fire scene and to escape from danger, these conventional fire escape systems often cannot help in a complex and constantly changing fire situation. Consequently, people caught in a fire often die from inhaling highly poisonous gases, or a lack of oxygen.

### SUMMARY OF THE INVENTION

Accordingly, an object of the present invention is to provide an emergency breathing tube that provides fresh air to people while they are waiting to be rescued from a fire, thereby reducing the mortality rate.

To attain the above object, the present invention provides an emergency breathing tube for fire victims, which breathing tube includes a first tube and a second tube. The first and second tubes couple together to form a retractile breathing tube with an adjustable length. When a fire occurs, a user can extend the breathing tube, hold one end of the breathing tube in the mouth, and then extend the other end thereof out from the smoky fire scene via an opening in the building to get fresh air. Thus, fir victims can survive a much longer time while waiting to be rescued, thereby reducing the mortality due to smoke inhalation.

### BRIEF DESCRIPTION OF THE DRAWINGS

Other objects, advantages, and novel features of the present invention will become more apparent from the following detailed description when taken in conjunction with the accompanying drawings, in which:

FIG. 1 is a perspective view of the first embodiment of the present invention;

FIG. 2 is a cross-sectional view of the first embodiment of the present invention;

FIG. 3 is a cross-sectional view of a retractile state of the first embodiment of the present invention;

FIG. 4 is a diagrammatic view showing a working state of the first embodiment of the present invention;

FIG. 5 is a cross-sectional view of the second embodiment of the present invention;

FIG. 6 is a cross-sectional view of a retractile state of the second embodiment of the present invention;

FIG. 7 is a perspective view of the third embodiment of the present invention;

FIG. 8 is a cross-sectional view of the third embodiment of the present invention;

FIG. 9 schematically shows a working state of the fourth embodiment of the present; and

FIG. 9A is a partial, enlarged view of FIG. 9.

### DETAILED DESCRIPTION OF PREFERRED EMBODIMENTS

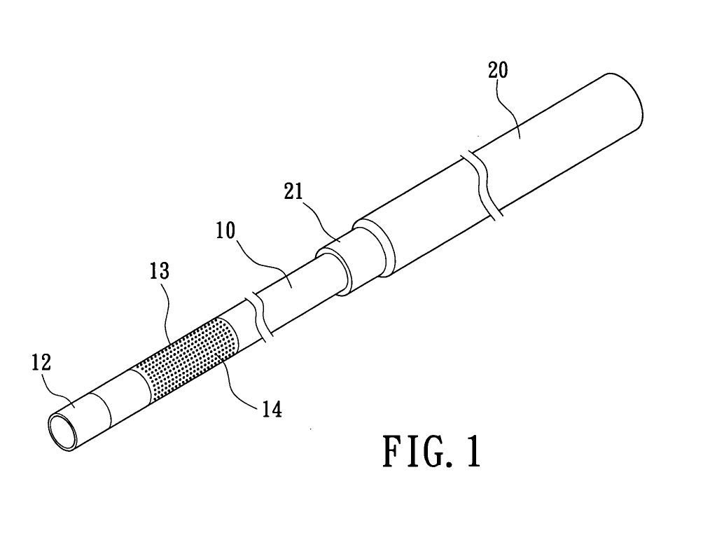

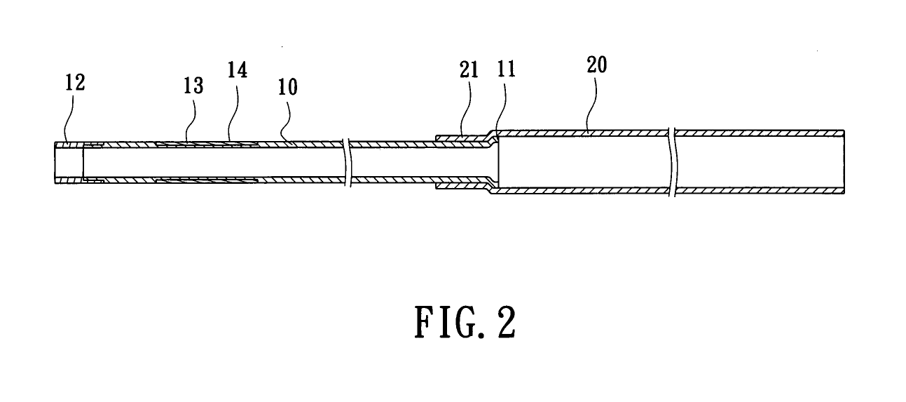

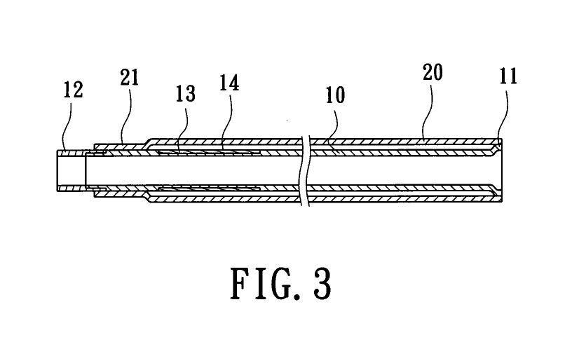

The present invention provides an emergency breathing tube for fire victims. FIG. 1 and FIG. 2 show the first embodiment of the present invention, which is a retractile breathing tube comprising a first tube 10 and a second tube 20. In order to withstand high temperatures in fire accident, the first tube 10 and the second tube 20 are made of a heat-resistant, easily molded material such as aluminum or the like, which is able to withstand a temperature of 300° C. The first tube 10 and the second tube 20 are hollow tubular bodies in a round shape or other suitable shapes and two opposite ends of each tube are open. An inner diameter of the second tube 20 is larger than an outer diameter of the first tube 10 so that the second tube 20 can contain the first tube 10. Then, the first tube 10 and the second tube 20 can couple together to form a retractile breathing tube, a length of which is adjustable. It is preferable that the breathing tube has a length of 2 meters or more when it is extended. Moreover, experiments show that a 4-meter-length breathing tube can still work quite well. Referring to FIG. 3, when not in use, the first tube 10 is retracted into the second tube 20 so as to reduce its length and save space.

The first tube 10 defines a locating portion 11 with a larger outer diameter at a back end thereof, and the second tube 20 defines a contractive portion 21 with a smaller outer diameter at a front end thereof. An inner wall of the contractive portion 21 of the second tube 20 contacts the outer wall of the first tube 10 closely so that the two tubes 10, 20 can maintain a perfect, airtight joint therebetween. When the first tube 10 and the second tube 20 are extended, the locating portion 11 abuts against the contractive portion 21. The first tube 10 and the second tube 20 are thereby located and prevented from being out of joint.

Selectively, the first tube 10 further includes a mouthpiece 12, which is hollow and is set on a front end of the first tube 10. A user can keep the mouthpiece 12 in his/her mouth during use. After use, the mouthpiece 12 is thrown away and is replaced with a new one for sanitation. If the mouthpiece 12 isn't provided, user can directly hold the front end of the first tube 10 in his or her mouth.

In addition, the present invention provides a holding portion 13 surrounding the outside of the first tube 10 for improving convenience of operation. An outer surface of the holding portion 13 is further defined as a studded surface 14, which could be made of raised stripes or lumps. The holding portion 13 is made of a heat-resistant material with low heat-conductivity, such as heat-resistant plastic. By the components that are described above, an emergency breathing tube for fire victims according the present invention is formed.

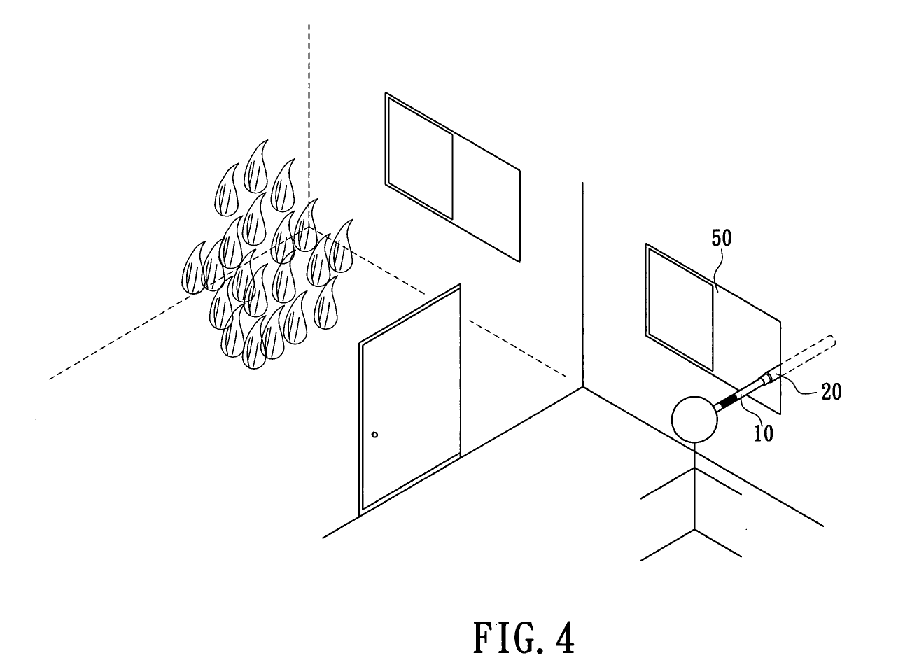

Referring to FIG. 4, when a fire occurs, a user can extend the breathing tube of the present invention, hold one end of the breathing tube in his or her mouth, and then extend the other end of the breathing tube out of the building via an opening 50 (for example, a window). In this way, the user can get fresh air from an outdoor location far away from the fire scene through the breathing tube (and exhale through the nose) so that the victim can survive a much longer time while waiting to be rescued, thereby reducing mortality due to smoke inhalation.

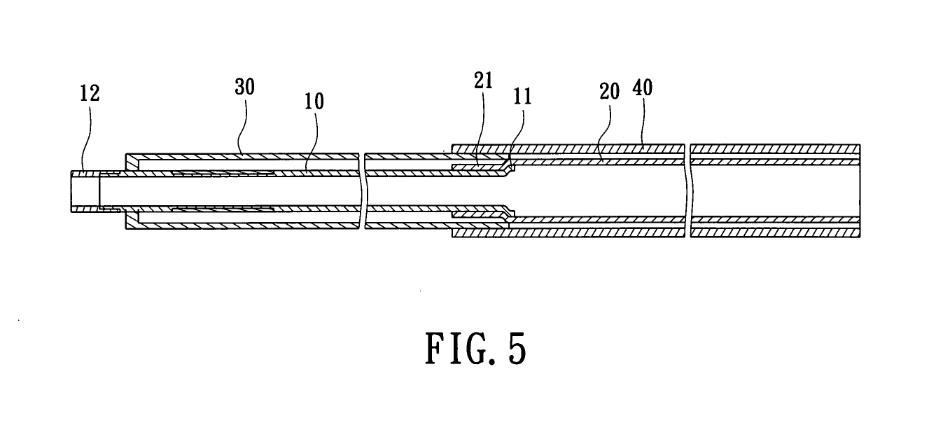

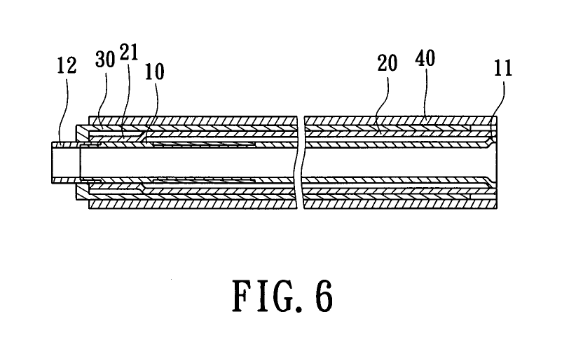

FIG. 5 and FIG. 6 show another embodiment of the breathing tube of the present invention, which further comprises a first outer tube 30 and a second outer tube 40, respectively disposed outside the first tube 10 and the second tube 20. The first outer tube 30 and the second outer tube 40 are made of a low heat-conductive material so that the user can hold the breathing tube with a hand but not be scalded, even when some part of the tube is heated by fire. Both the first outer tube 30 and the second outer tube 40 are round hollow tubes, and their inner diameters are greater than the outer diameters of the first tube 10 and the second tube 20. Furthermore, the inner diameter of the second outer tube 40 is greater than an outer diameter of the first outer tube 30 so that the first outer tube 30 can be contained in the second outer tube 40. As a result, the first outer tube 30 and the second outer tube 40 also can couple together to form a retractile outer tube with an adjustable length so as to keep a synchronous retractile motion with respect to that of the first tube 10 and the second tube 20. However, the first outer tube 30 and the second outer tube 40 also can be designed as hollow tubes with other shapes (not shown). The operation method and function of the present embodiment are similar to those of the first embodiment, and thus are not further described here.

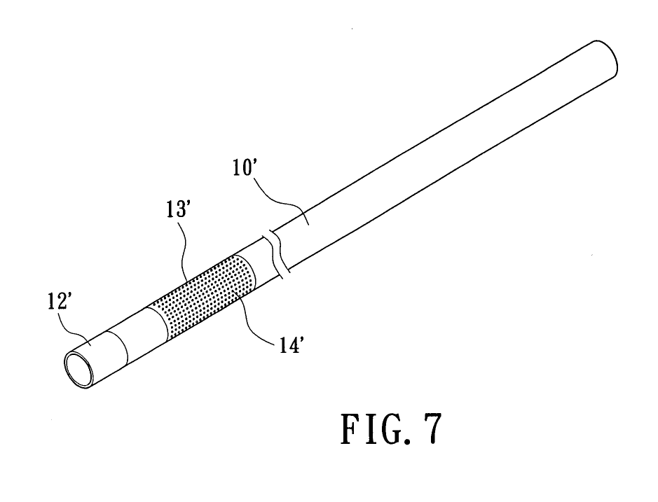

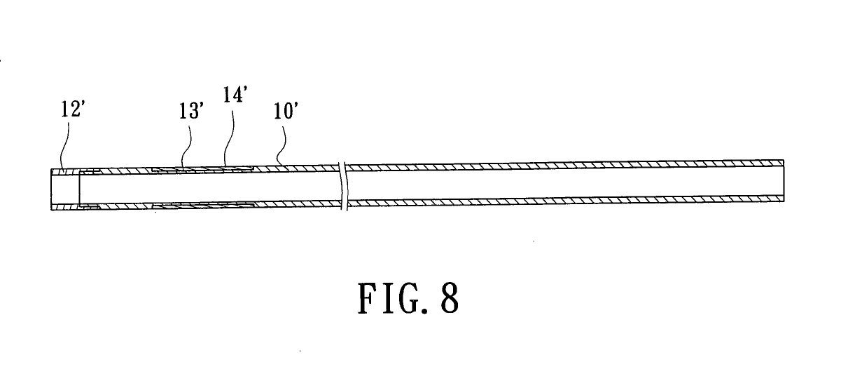

Referring to FIG. 7 and FIG. 8, this embodiment is a breathing tube having a fixed length and only comprising a first tube 10′. The first tube 10′ is made of a material with perfect heat-resistant characteristics. The first tube 10′ is a hollow tubular body in a round shape or other shapes and two opposite ends thereof are open. The first tube 10′ also provides a replaceable mouthpiece 12′ selectively applied on a front end thereof. A holding portion 13′ is positioned outside the first tube 10′, which is made of a low heat-conductive material and has a studded surface 14′ consisting of raised stripes or lumps. Furthermore, the first tube 10′ also has an outer tube (not shown), which is disposed outside the first tube 10′ and is made of a low heat-conductive material. In addition, the breathing tube can be designed to have a fluorescent coating for help alert. The operation method and function of the present embodiment are similar to those of the first embodiment, and thus not further described here.

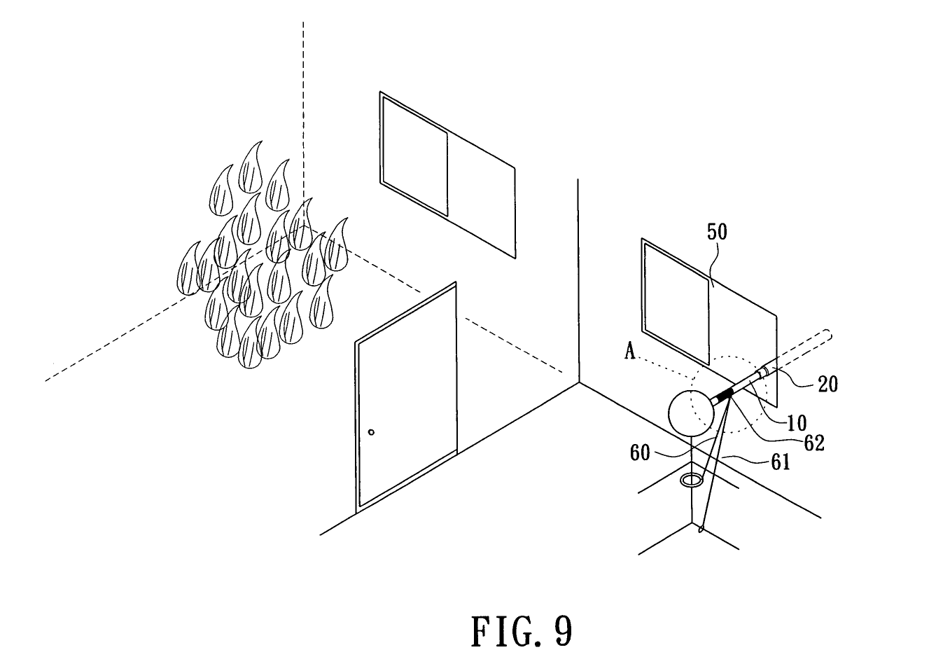

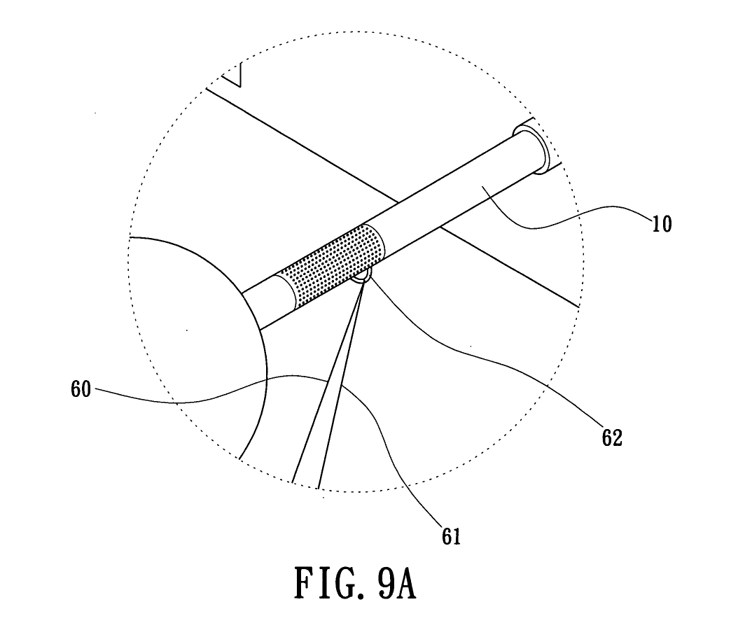

Referring to FIG. 9 and FIG. 9A, a breathing tube of this embodiment is an improvement of the first embodiment that further comprises a pair of safety belts 60, 61, which have enough resistance to resist external pulling forces and are adjustable in length. One end of each safety belt 60, 61 is hooked over and fastened on a hooking ring 62 integrally formed on the first tube 10, the other end of the safety belt 60 is fastened on the user's body (chest or wrist), and the other end to the safety belt 61 is placed around the user's leg (or fixed by the user's foot). Moreover, the safety belt 60 fastened on the user's body is designed as a rather wide band. In use, the user can put the breathing tube onto the bottom of the opening 50 of a building as a fulcrum for labor-saving according to the principle of leverage.

In other words, the tractable emergency breathing tube includes a first tube 10 and a second tube 20 assembled together to form a series of telescoping tubes. The telescoping tubes are nested one within the other when the emergency breathing tube in a retracted stowed position. The inner tubes are latched in the stowed position by a caging mechanism.

Furthermore, the emergency breathing tube can be extended until the final deployed position to reached out of the building on fire for fresh air. A user can inhale fresh air through the pursed mouth slowly by means of the instrument and exhale carbon dioxide through the nose rapidly, and repeat the above inhale/exhale procedures until being rescued.

It is to be understood, however, that even though numerous characteristics and advantages of the present invention have been set forth in the foregoing description, together with details of the structure and function of the invention, the disclosure is illustrative only, and changes may be made in detail, especially in matters of shape, size and arrangement of parts within the principles of the invention to the full extent indicated by the broad general meaning of the terms in which the appended claims are expressed.

1. An emergency breathing tube for life-saving in case of fire, comprising:
a first tube; and
a second tube, wherein the second tube couples with the first tube and forms a retractile breathing tube with adjustable length;
wherein, in case of fire, a user extends the breathing tube, holds one end of the breathing tube in his or her mouth, and then extends another end thereof out from the fire scene via an opening of a building to get fresh air.

2. The emergency breathing tube for life-saving in case of fire as claimed in claim 1, wherein the first and second tubes are made of material with perfect heat-resistant characteristics.

3. The emergency breathing tube for life-saving in case of fire as claimed in claim 1, wherein an inner diameter of the second tube is larger than an outer diameter of the first tube, and the second tube is mounted outside the first tube.

4. The emergency breathing tube for life-saving in case of fire as claimed in claim 1, wherein one end of the first tube defines a locating portion, and one end of the second tube defines a contractive portion, an inner wall of the contractive portion contacts an outer wall of the first tube closely, and the locating portion abuts against the contractive portion, the first tube and the second tube thereby being located when they are extended.

5. The emergency breathing tube for life-saving in case of fire as claimed in claim 1, wherein the first tube has a replaceable mouthpiece mounted on an end thereof.

6. The emergency breathing tube for life-saving in case of fire as claimed in claim 1, wherein the first tube is ringed with a holding portion, and the holding portion is made of a low heat-conductive material and has an studded surface.

7. The emergency breathing tube for life-saving in case of fire as claimed in claim 1, further comprising a first outer tube and a second outer tube respectively disposed outside the first tube and the second tube, wherein the first outer tube and the second outer tube are made of a low heat-conductive material and capable of coupling together to form a retractile outer tube with an adjustable length.

8. The emergency breathing tube for life-saving in case of fire as claimed in claim 1, further comprising a pair of safety belts fastened thereon, wherein the safety belts have enough resistance to resist external pulling forces and are adjustable in length, and one end of the rope is fastened to the breathing tube while another end thereof is fastened to the user.

9. An emergency breathing tube for life-saving in case of fire, comprising a tubular body made of a material with perfect heat-resistance; whereby, when a fire occurs, a user holds one end of the breathing tube in his or her mouth, and extends another end thereof out from the smoky fire scene via an opening in a building to get fresh air.

10. The emergency breathing tube for life-saving in case of fire as claimed in claim 9, wherein the tubular body has a replaceable mouthpiece mounted on an end thereof.

11. The emergency breathing tube for life-saving in case of fire as claimed in claim 9, wherein the tubular body is ringed with a holding portion, and the holding portion is made of a low heat-conductive material and has an studded surface.

12. The emergency breathing tube for life-saving in case of fire as claimed in claim 9, further comprising an outer tube made of a low heat-conductive material disposed outside the tubular body.

13. The emergency breathing tube for life-saving in case of fire as claimed in claim 9, further comprising a pair of safety belts fastened thereon, wherein the safety belts have enough resistance to resist external pulling forces and are adjustable in length, and one end of the rope is fastened to the breathing tube while another end thereof is fastened to the user.
